(19) United States
(12) Patent Application Publication
(10) Pub. No.: US 2012/0129234 A1
(43) Pub. Date: May 24, 2012

(54) SYSTEM FOR TREATMENT OF BIOMASS TO FACILITATE THE PRODUCTION OF ETHANOL

(75) Inventors: William F. McDonald, Utica, OH (US); Nicholas Paul Stutzman, Yankton, SD (US); David Charles Carlson, Yankton, SD (US)

(73) Assignee: POET Research, Inc., Sioux Falls, SD (US)

(21) Appl. No.: 13/300,244

(22) Filed: Nov. 18, 2011

(30) Foreign Application Priority Data
May 18, 2010 (US) PCT/US10/35316

(51) Int. Cl. C12P 7/10; C12M 1/16

(52) U.S. Cl. 435/165; 435/289.1

(57) ABSTRACT

A method for treating fermented lignocellulosic biomass to be supplied to a distillation system for production of ethanol is provided. The method includes pre-treating lignocellulosic biomass into pre-treated biomass and separating the pre-treated biomass into a liquid component comprising sugars and a solids component comprising cellulose and lignin. The method also includes hydrolysing the solids component of the pre-treated biomass into a hydrolysed biomass comprising sugars and lignin and fermenting the hydrolysed solids component of the pre-treated biomass into a fermentation product comprising ethanol and lignin. The method also includes treating the fermentation product and distilling the treated fermentation product to recover the ethanol. The lignocellulosic biomass comprises cellulose, hemi-cellulose and lignin.

### CROSS REFERENCE TO RELATED APPLICATIONS

This application is a U.S. national stage filing of Patent Cooperation Treaty (PCT) application serial number PCT/US10/35316 entitled “SYSTEM FOR TREATMENT OF BIOMASS TO FACILITATE THE PRODUCTION OF ETHANOL” filed on May 18, 2010, which claims the benefit of U.S. Provisional Application Ser. No. 61/179,349, titled “DISTILLATION OF HEXOSE BEER”, filed on May 18, 2009. The entireties of the aforementioned applications are herein incorporated by reference.

### FIELD

The present invention relates to a system for treatment of biomass in the production of ethanol. The present invention also relates to a system for treatment of fermented biomass before the fermented biomass is supplied to a distillation system in order to facilitate the efficient production of ethanol.

### BACKGROUND

Ethanol can be produced from grain-based feedstocks (e.g. corn, sorghum/milo, barley, wheat, soybeans, etc.), from sugar (e.g. from sugar cane, sugar beets, etc.), and from biomass (e.g. from lignocellulosic feedstocks such as switchgrass, corn cobs and stover, wood or other plant material).

Biomass comprises plant matter that can be suitable for direct use as a fuel/energy source or as a feedstock for processing into another bioproduct (e.g., a biofuel such as cellulosic ethanol) produced at a biorefinery (such as an ethanol plant). Biomass may comprise, for example, corn cobs and stover (e.g., stalks and leaves) made available during or after harvesting of the corn kernels, fiber from the corn kernel, switchgrass, farm or agricultural residue, wood chips or other wood waste, and other plant matter). In order to be used or processed, biomass will be harvested and collected from the field and transported to the location where it is to be used or processed.

In a conventional ethanol plant producing ethanol from corn, ethanol is produced from starch. Corn kernels may be processed to separate the starch-containing material (e.g. endosperm) from other matter (such as fiber and germ). The starch-containing material is slurried with water and liquefied to facilitate saccharification where the starch is converted into sugar (e.g. glucose) and fermentation where the sugar is converted by an ethanologen (e.g. yeast) into ethanol. The product of fermentation (e.g. fermentation product) is beer, which comprises a liquid component containing ethanol and water (among other things) and a solids component containing unfermented particulate matter (among other things). The liquid component and solids component of the fermentation product is sent to a distillation system. In the distillation system, the fermentation product is distilled and dehydrated into, among other things, ethanol and stillage containing wet solids (e.g., the solids component of the beer with substantially all ethanol removed) that can be dried into dried distillers grains (DDG) and sold as an animal feed product. Other co-products, for example syrup (and oil contained in the syrup), can also be recovered from the stillage. Water removed from the fermentation product in distillation can be treated for re-use at the plant.

In a biorefinery configured to produce ethanol from biomass such as cellulosic feedstocks, ethanol is produced from lignocellulosic material (e.g., cellulose and/or hemi-cellulose). The biomass is prepared so that sugars in the cellulosic material (such as glucose from the cellulose and xylose from the hemi-cellulose) can be accessed and fermented into a fermentation product that comprises ethanol (among other things). The fermentation product is then sent to the distillation system, where the ethanol is recovered by distillation and dehydration. Other bioproducts such as lignin and organic acids may also be recovered as co-products. Determination of how to more efficiently prepare and treat the biomass for production into ethanol will depend upon (among other things) the form, type and composition of the biomass.

It would be advantageous to provide for a system for treating biomass in the production of ethanol. It would further be advantageous to provide for a system for treating biomass before the biomass is supplied to a distillation system.

### SUMMARY

The present invention relates to a method for treating fermented lignocellulosic biomass to be supplied to a distillation system for production of ethanol. The method includes pre-treating lignocellulosic biomass into pre-treated biomass and separating the pre-treated biomass into a liquid component comprising sugars and a solids component comprising cellulose and lignin. The method also includes hydrolysing the solids component of the pre-treated biomass into a hydrolysed biomass comprising sugars and lignin and fermenting the hydrolysed solids component of the pre-treated biomass into a fermentation product comprising ethanol and lignin. The method also includes treating the fermentation product and distilling the treated fermentation product to recover the ethanol. The lignocellulosic biomass comprises cellulose, hemi-cellulose and lignin.

The present invention also relates to a system for treating fermented lignocellulosic biomass. The system comprises a pre-treatment system that creates pre-treated biomass from lignocellulosic biomass and a separation system that separates the pre-treated biomass into a liquid component comprising sugars and a solids component comprising cellulose and lignin. The system also comprises a first treatment system that hydrolyses the solids component of the pre-treated biomass into a hydrolysed biomass comprising sugars and lignin and a fermentation system that ferments the hydrolysed solids component of the pre-treated biomass into a fermentation product comprising ethanol and lignin. Further, the system comprises a second treatment system that treats the fermentation product and a distillation system that distills the treated fermentation product to recover the ethanol. According to some embodiments, the second treatment system heats the fermentation product.

### DESCRIPTION OF THE DRAWINGS

FIG. 1A is a perspective view of a biorefinery comprising a cellulosic ethanol production facility.

FIG. 1B is a perspective view of a biorefinery comprising a cellulosic ethanol production facility and a corn-based ethanol production facility.

FIG. 2 is a schematic diagram of a system for receipt and preparation of biomass for a cellulosic ethanol production facility.

FIG. 3 is a schematic block diagram of a system for the production of ethanol from biomass.

FIGS. 4A, 4B and 4C are schematic block diagrams of systems for treatment and processing of components from the production of ethanol from biomass.

FIG. 5A is a schematic block diagram of an apparatus used for preparation, pre-treatment and separation of biomass.

FIG. 5B is a perspective view of an apparatus used to pre-treat and separate the biomass.

FIGS. 6A and 6B are schematic diagrams of the process flow for systems for the production of ethanol from biomass.

FIGS. 7A and 7B are schematic block diagrams of a treatment system according to an exemplary embodiment.

FIG. 8 is a schematic block diagram of a treatment system according to an exemplary embodiment.

FIG. 9A is a perspective view of a distillation column.

FIG. 9B is a schematic diagram of a distillation column.

FIG. 10 is a schematic view of a sieve tray.

FIG. 11 is a graph showing the effect of distillation trays and treatment of distillation runtime.

FIG. 12 is a graph showing the effect of treatment on the particle size of the fermentation product.

FIGS. 13A, 13B, and 13C are graphical presentations of typical operating conditions of a treatment system.

TABLES 1A and 1B list the composition of biomass comprising lignocellulosic plant material from the corn plant according to exemplary and representative embodiments.

TABLES 2A and 2B list the composition of the liquid component of pre-treated biomass according to exemplary and representative embodiments.

TABLES 3A and 3B list the composition of the solids component of pre-treated biomass according to exemplary and representative embodiments.

TABLES 4A and 4B list the average composition of the fermentation product.

### DESCRIPTION OF THE EMBODIMENTS

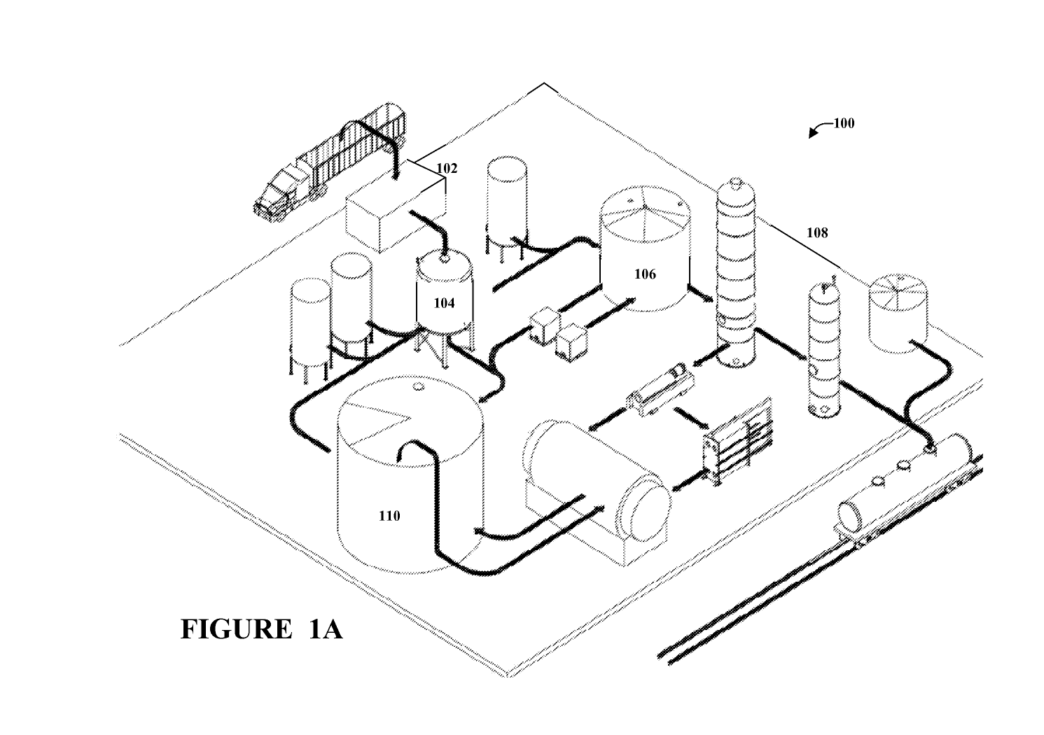

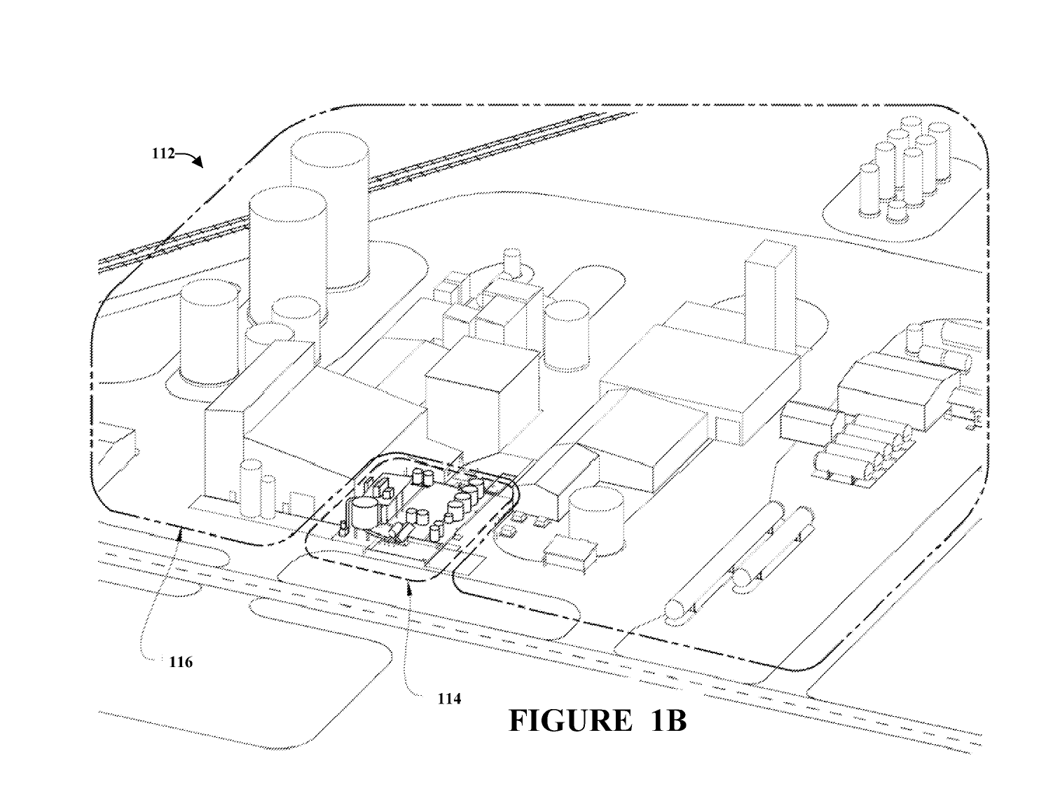

Referring to FIG. 1A, a biorefinery 100 configured to produce ethanol from biomass is shown.

According to an exemplary embodiment, the biorefinery 100 is configured to produce ethanol from biomass in the form of a lignocellulosic feedstock such as plant material from the corn plant (e.g. corn cobs and corn stover). Lignocellulosic feedstock such as lignocellulosic material from the corn plant comprises cellulose (from which C6 sugars such as glucose can be made available) and/or hemicellulose (from which C5 sugars such as xylose and arabinose can be made available).

As shown in FIG. 1A, the biorefinery comprises an area where biomass is delivered and prepared to be supplied to the cellulosic ethanol production facility. The cellulosic ethanol production facility comprises apparatus for preparation 102, pre-treatment 104 and treatment of the biomass into treated biomass suitable for fermentation into fermentation product in a fermentation system 106. The facility comprises a distillation system 108 in which the fermentation product is distilled and dehydrated into ethanol. As shown in FIG. 1A, the biorefinery may also comprise a waste treatment system 110 (shown as comprising an anaerobic digester and a generator). According to other alternative embodiments, the waste treatment system may comprise other equipment configured to treat, process and recover components from the cellulosic ethanol production process, such as a solid/waste fuel boiler, anaerobic digester, aerobic digester or other biochemical or chemical reactors.

As shown in FIG. 1B, according to an exemplary embodiment, a biorefinery 112 may comprise a cellulosic ethanol production facility 114 (which produces ethanol from lignocellulosic material and components of the corn plant) co-located with a corn-based ethanol production facility 116 (which produces ethanol from starch contained in the endosperm component of the corn kernel). As indicated in FIG. 1B, by co-locating the two ethanol production facilities, certain plant systems may be shared, for example, systems for dehydration, storage, denaturing and transportation of ethanol, energy/fuel-to-energy generation systems, plant management and control systems, and other systems. Corn fiber (a component of the corn kernel), which can be made available when the corn kernel is prepared for milling (e.g. by fractionation) in the corn-based ethanol production facility, may be supplied to the cellulosic ethanol production facility as a feedstock. Fuel or energy sources such as methane or lignin from the cellulosic ethanol production facility may be used to supply power to either or both co-located facilities. According to other alternative embodiments, a biorefinery (e.g. a cellulosic ethanol production facility) may be co-located with other types of plants and facilities, for example an electric power plant, a waste treatment facility, a lumber mill, a paper plant or a facility that processes agricultural products.

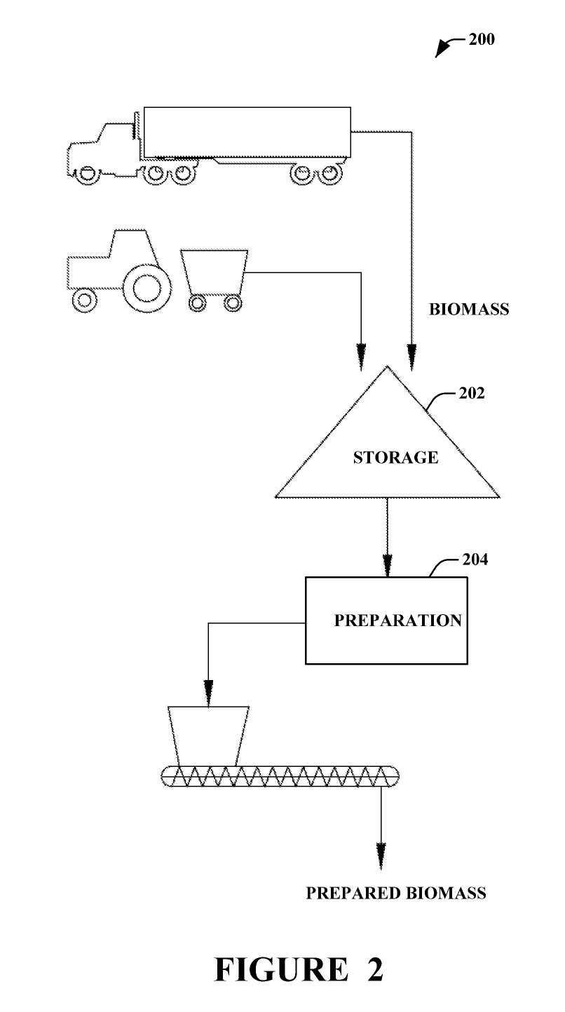

Referring to FIG. 2, a system 200 for preparation of biomass delivered to the biorefinery is shown. The biomass preparation system may comprise apparatus for receipt/unloading of the biomass, cleaning (e.g. removal of foreign matter), grinding (e.g. milling, reduction or densification), and transport and conveyance for processing at the plant. According to an exemplary embodiment, biomass in the form of corn cobs and stover may be delivered to the biorefinery and stored 202 (e.g. in bales, piles or bins, etc.) and managed for use at the facility. According to a preferred embodiment, the biomass may comprise at least 20 to 30 percent corn cobs (by weight) with corn stover and other matter. According to other exemplary embodiments, the preparation system 204 of the biorefinery may be configured to prepare any of a wide variety of types of biomass (e.g. plant material) for treatment and processing into ethanol and other bioproducts at the plant.

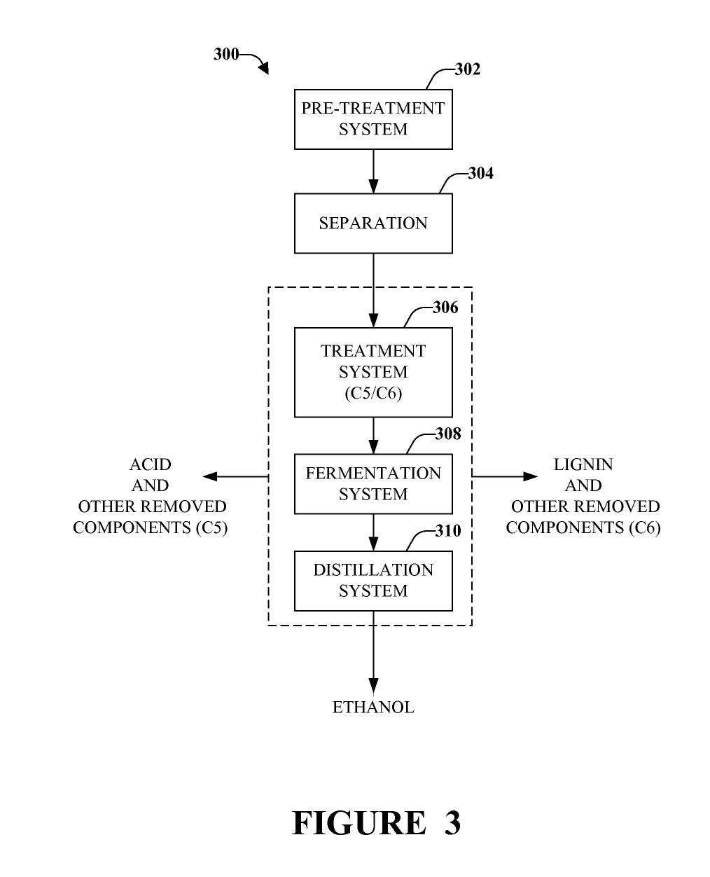

Referring to FIG. 3, a schematic diagram of the cellulosic ethanol production facility 300 is shown. According to a preferred embodiment, biomass comprising plant material from the corn plant is prepared and cleaned at a preparation system. After preparation, the biomass is mixed with water into a slurry and is pre-treated at a pre-treatment system 302. In the pre-treatment system 302, the biomass is broken down (e.g. by hydrolysis) to facilitate separation 304 into a liquid component (e.g. a stream comprising the C5 sugars) and a solids component (e.g. a stream comprising cellulose from which the C6 sugars can be made available). The C5-sugar-containing liquid component (C5 stream) and C6-sugar-containing solids component (C6 stream) can be treated in a treatment system 306 (as may be suitable) and fermented in a fermentation system 308. Fermentation product from the fermentation system 308 is supplied to a distillation system 310 where the ethanol is recovered.

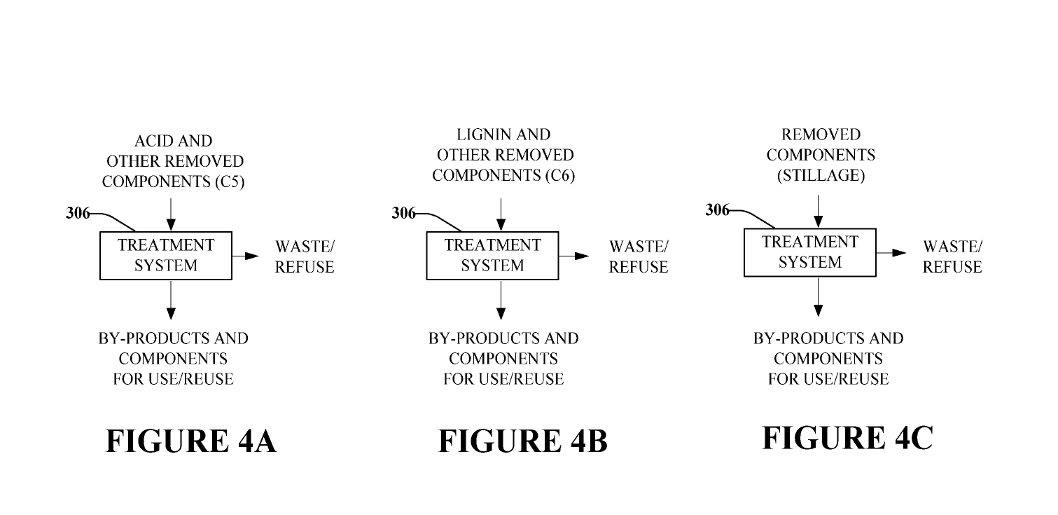

As shown in FIGS. 3 and 4A, removed components from treatment of the C5 stream can be treated or processed to recover by-products, such as organic acids, furfural, and lignin. The removed components during treatment and production of ethanol from the biomass from either or both the C5 stream and the C6 stream (or at distillation) can be treated or processed into bioproducts or into fuel (such as lignin for a solid fuel boiler or methane produced by treatment of residual/removed matter such as acids and lignin in an anaerobic digester) or recovered for use or reuse.

According to a preferred embodiment, the biomass comprises plant material from the corn plant, such as corn cobs, husks and leaves and stalks (e.g. at least upper half or three-quarters portion of the stalk); the composition of the plant material (e.g. cellulose, hemicellulose and lignin) will be approximately as indicated in TABLES 1A and 1B (e.g. after at least initial preparation of the biomass, including removal of any foreign matter). According to a preferred embodiment, the plant material comprises corn cobs, husks/leaves and stalks; for example, the plant material may comprise (by weight) up to 100 percent cobs, up to 100 percent husks/leaves, approximately 50 percent cobs and approximately 50 percent husks/leaves, approximately 30 percent cobs and approximately 50 percent husks/leaves and approximately 20 percent stalks, or any of a wide variety of other combinations of cobs, husks/leaves and stalks from the corn plant. See TABLE 1A. According to an alternative embodiment, the lignocellulosic plant material may comprise fiber from the corn kernel (e.g. in some combination with other plant material). TABLE 1B provides typical and expected ranges believed to be representative of the composition of biomass comprising lignocellulosic material from the corn plant. According to exemplary embodiments, the lignocellulosic plant material of the biomass (from the corn plant, such as corn cobs, corn plant husks, corn plant leaves, and corn stalks) will comprise (by weight) cellulose at about 30 to 55 percent by weight, hemicellulose at about 20 to 50 percent by weight, and lignin at about 10 to 25 percent by weight; according to a particularly preferred embodiment, the lignocellulosic plant material of the biomass (e.g. cobs, husks/leaves and stalk portions from the corn plant) will comprise (by weight) cellulose at about 35 to 45 percent by weight, hemicellulose at about 24 to 42 percent by weight, and lignin at about 12 to 20 percent by weight. According to a particularly preferred embodiment, pre-treatment of the biomass will yield a liquid component that comprises (by weight) xylose at no less than 1.0 percent and a solids component that comprises (by weight) cellulose (from which glucose can be made available) at no less than 45 percent.

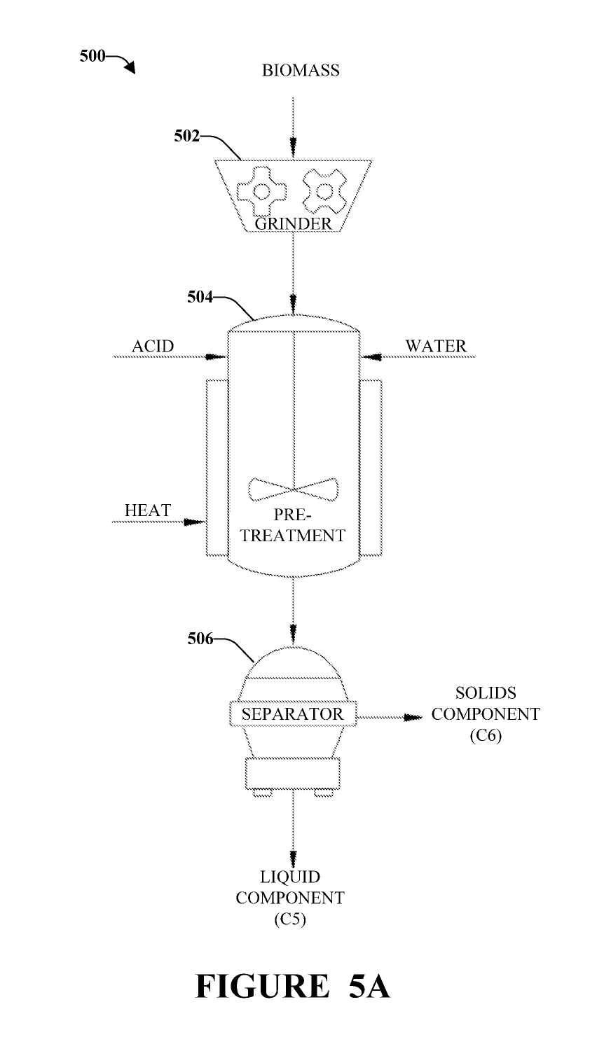

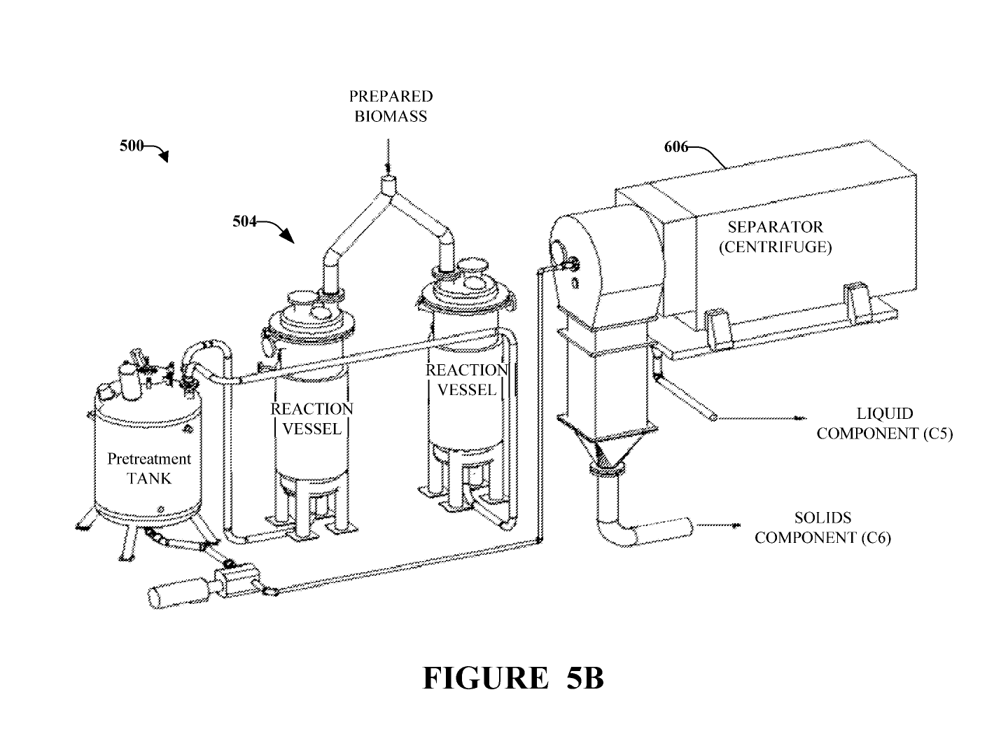

FIGS. 5A and 5B show the apparatus 500 used for preparation, pre-treatment and separation of lignocellulosic biomass according to an exemplary embodiment. As shown, biomass is prepared in a grinder 502 (e.g. grinder or other suitable apparatus or mill). Pre-treatment 504 of the prepared biomass is performed in a reaction vessel (or set of reaction vessels) supplied with prepared biomass and acid/water in a predetermined concentration (or pH) and other operating conditions. As shown in FIG. 5B, the pre-treated biomass can be separated in a centrifuge 506 into a liquid component (C5 stream comprising primarily liquids with some solids) and a solids component (C6 stream comprising liquids and solids such as lignin and cellulose from which glucose can be made available by further treatment).

According to a preferred embodiment, in the pre-treatment system an acid will be applied to the prepared biomass to facilitate the breakdown of the biomass for separation into the liquid component (C5 stream from which fermentable C5 sugars can be recovered) and the solids component (C6 stream from which fermentable C6 sugars can be accessed). According to a preferred embodiment, the acid can be applied to the biomass in a reaction vessel under determined operating conditions (e.g. acid concentration, pH, temperature, time, pressure, solids loading, flow rate, supply of process water or steam, etc.) and the biomass can be agitated/mixed in the reaction vessel to facilitate the break down of the biomass. According to exemplary embodiments, an acid such as sulfuric acid, hydrochloric acid, nitric acid, phosphoric acid, acetic acid, etc. (or a formulation/mixture of acids) can be applied to the biomass. According to a particularly preferred embodiment, sulfuric acid will be applied to the biomass in pre-treatment.

The liquid component (C5 stream) comprises water, dissolved sugars (such as xylose, arabinose and glucose) to be made available for fermentation into ethanol, acids and other soluble components recovered from the hemicellulose. (TABLE 2B provides typical and expected ranges believed to be representative of the composition of biomass comprising lignocellulosic material from the corn plant.) According to an exemplary embodiment, the liquid component may comprise approximately 5 to 7 percent solids (e.g. suspended/residual solids such as partially-hydrolysed hemicellulose, cellulose and lignin). According to a particularly preferred embodiment, the liquid component will comprise at least 2 to 4 percent xylose (by weight); according to other exemplary embodiments, the liquid component will comprise no less than 1 to 2 percent xylose (by weight). TABLES 2A and 2B list the composition of the liquid component of pre-treated biomass (from prepared biomass as indicated in TABLES 1A and 1B) according to exemplary and representative embodiments.

The solids component (C6 stream) comprises water, acids and solids such as cellulose from which sugar, such as glucose, can be made available for fermentation into ethanol, and lignin. (TABLE 3B provides typical and expected ranges believed to be representative of the composition of biomass comprising lignocellulosic material from the corn plant.) According to an exemplary embodiment, the solids component may comprise approximately 10 to 40 percent solids (by weight) (after separation); according to a particularly preferred embodiment, the solids component will comprise approximately 20 to 30 percent solids (by weight). According to a preferred embodiment, the solids in the solids component comprise no less than 30 percent cellulose and the solids component may also comprise other dissolved sugars (e.g. glucose and xylose). TABLES 3A and 3B list the composition of the solids component of pre-treated biomass (from prepared biomass as indicated in TABLES 1A and 1B) according to exemplary and representative embodiments.

During pre-treatment, the severity of operating conditions (such as pH, temperature and time) may cause formation of components that are inhibitory to fermentation. For example, under some conditions, the dehydration of C5 sugars (such as xylose or arabinose) may cause the formation of furfural. Acetic acid may also be formed, for example, when acetate is released during the break down of hemicellulose in pre-treatment. Sulfuric acid, which may be added to prepared biomass to facilitate pre-treatment, if not removed or neutralized, may also be inhibitory to fermentation. According to an exemplary embodiment, by adjusting pre-treatment conditions (such as pH, temperature and time), the formation of inhibitors can be reduced or managed; according to other exemplary embodiments, components of the pre-treated biomass may be given further treatment to remove or reduce the level of inhibitors (or other undesirable matter).

According to an exemplary embodiment, biomass can be pre-treated by mixing prepared biomass with water and acid (e.g. sulfuric acid) to form a slurry that comprises approximately 10 to 30 percent solids (by weight) from the biomass and approximately 0.8 to 1.3 percent acid (by weight). The temperature of the slurry is held at approximately 130 to 185 degrees Celsius for about 3 to 15 minutes. According to an embodiment, the pre-treatment is conducted in a closed reaction vessel at a pressure that may increase during the reaction from ambient pressure to approximately 100 to 120 pounds per square inch.

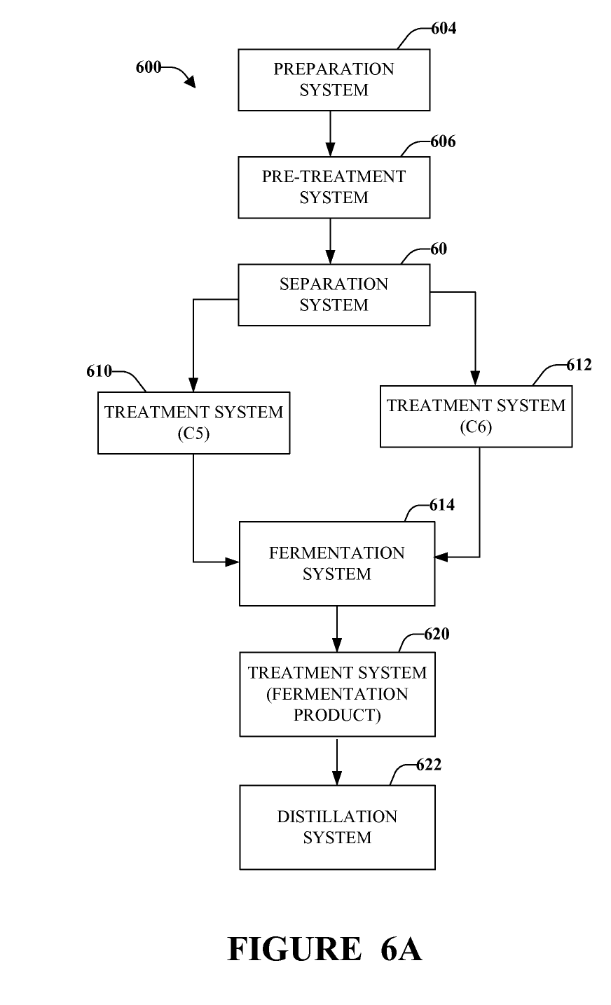

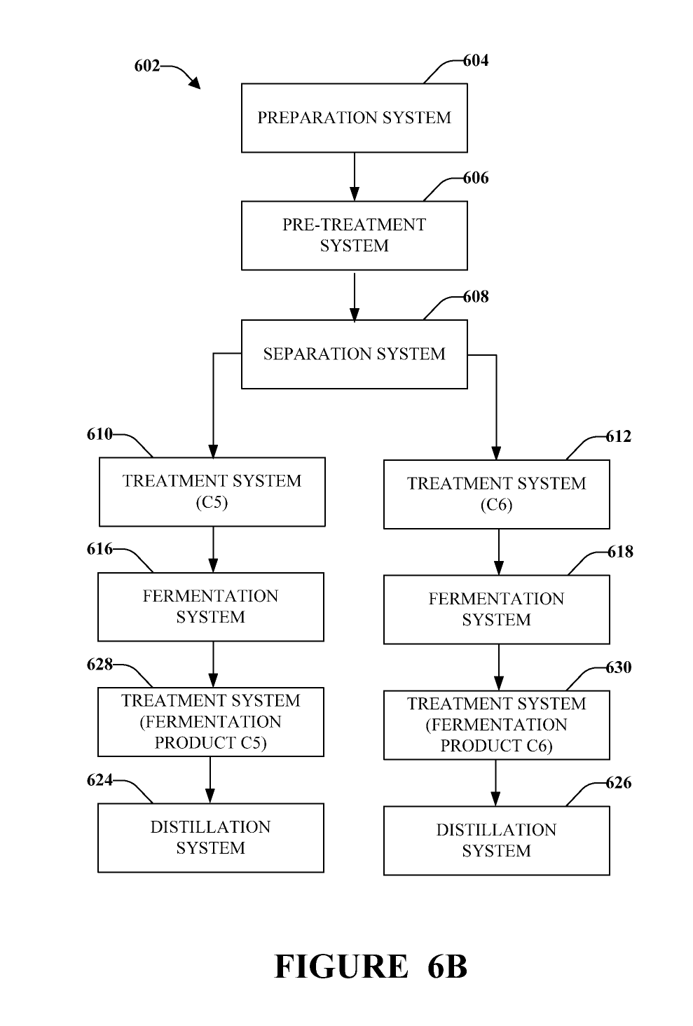

Referring to FIGS. 6A and 6B, after pre-treatment and separation the C5 stream and the C6 stream are processed separately; as shown, the C5 stream and the C6 stream may be processed separately (in separate treatment systems 610, 612) prior to co-fermentation 614 (C5/C6 fermentation as shown in FIG. 6A) or processed separately (in separate treatment systems 610, 612) including separate fermentation (separate C5 fermentation and C6 fermentation 616, 618 as shown in FIG. 6B).

Treatment of the C5 stream (liquid component) of the biomass may be performed in an effort to remove components that are inhibitory to efficient fermentation (e.g. furfural, hydroxymethylfurfural (HMF), sulfuric acid and acetic acid) and residual lignin (or other matter) that may not be fermentable from the C5 sugar component so that the sugars (e.g. xylose, arabinose, as well as other sugars such as glucose) are available for fermentation. The C5 sugars in the C5 stream may also be concentrated to improve the efficiency of fermentation (e.g. to improve the titer of ethanol for distillation).

Treatment of the C6 stream (solids component) of the biomass may be performed to make the C6 sugars available for fermentation. According to a preferred embodiment, hydrolysis (such as enzyme hydrolysis) may be performed to access the C6 sugars in the cellulose; treatment may also be performed in an effort to remove lignin and other non-fermentable components in the C6 stream (or to remove components such as residual acid or acids that may be inhibitory to efficient fermentation).

According to an exemplary embodiment shown in FIG. 6A, after pre-treatment and separation the C5 stream and the C6 stream can be treated separately and subsequently combined after treatment (e.g. as a slurry) for co-fermentation 614 in the fermentation system to produce a C5/C6 fermentation product from the available sugars (e.g. xylose and glucose); the C5/C6 fermentation product can (after treatment 620, if any) be supplied to the distillation system 622 for recovery of the ethanol (e.g. through distillation and dehydration). According to an exemplary embodiment shown in FIG. 6B, the C5 stream and the C6 stream can each be separately processed through fermentation 616, 618 and distillation 624, 626 (after treatment 628, 630, if any) to produce ethanol. According to any preferred embodiment, a suitable fermenting organism (ethanologen) will be used in the fermentation system; the selection of an ethanologen may be based on various considerations, such as the predominant types of sugars present in the slurry. Dehydration and/or denaturing of the ethanol produced from the C5 stream and the C6 stream may be performed either separately or in combination.

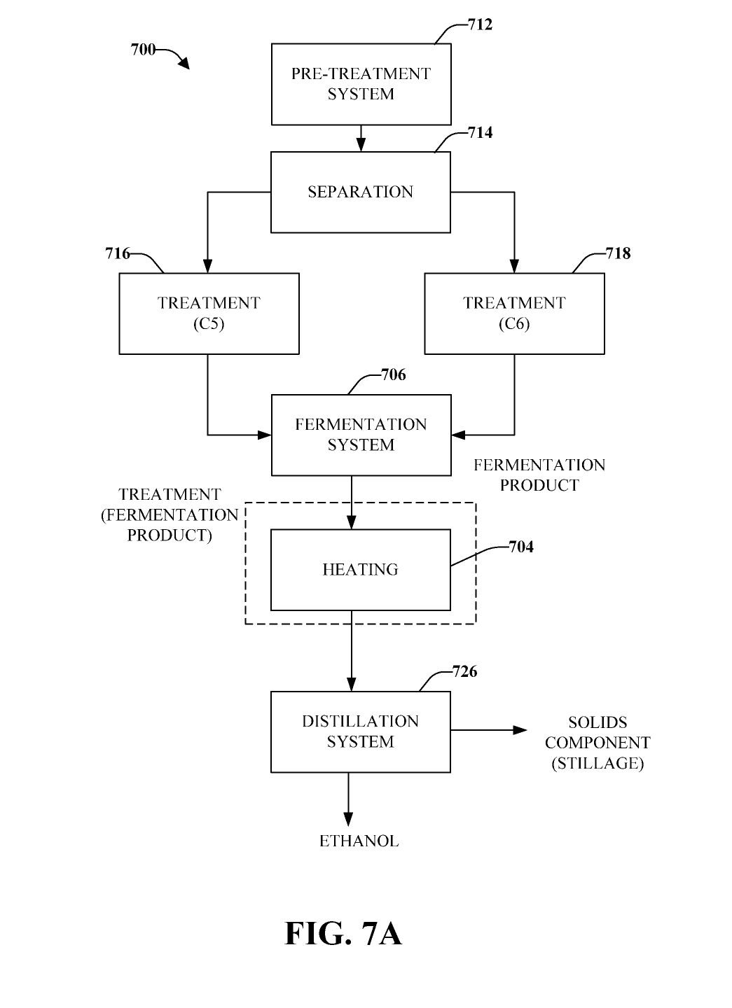

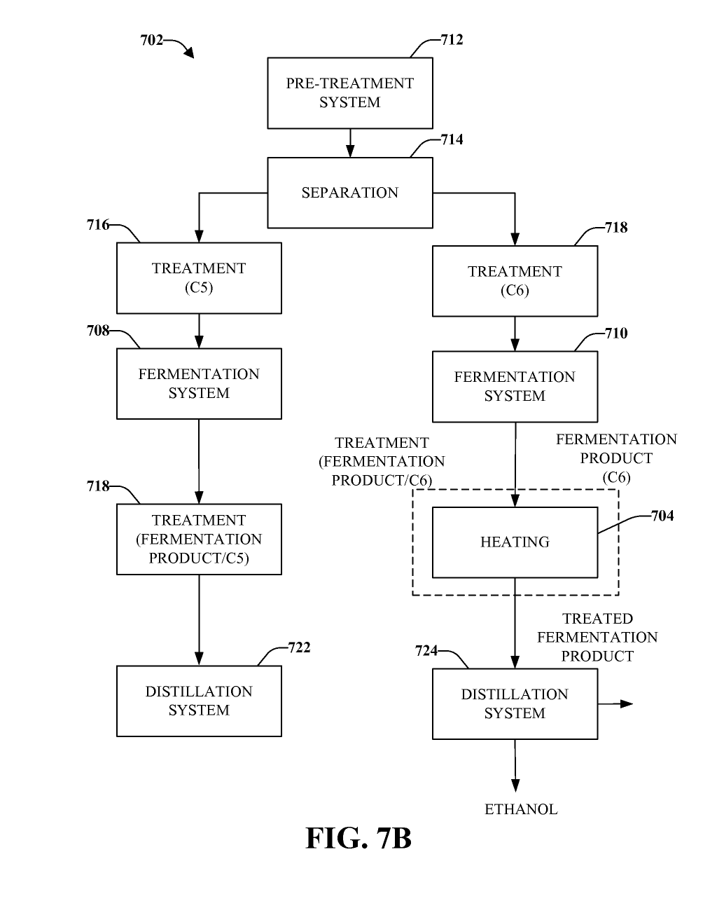

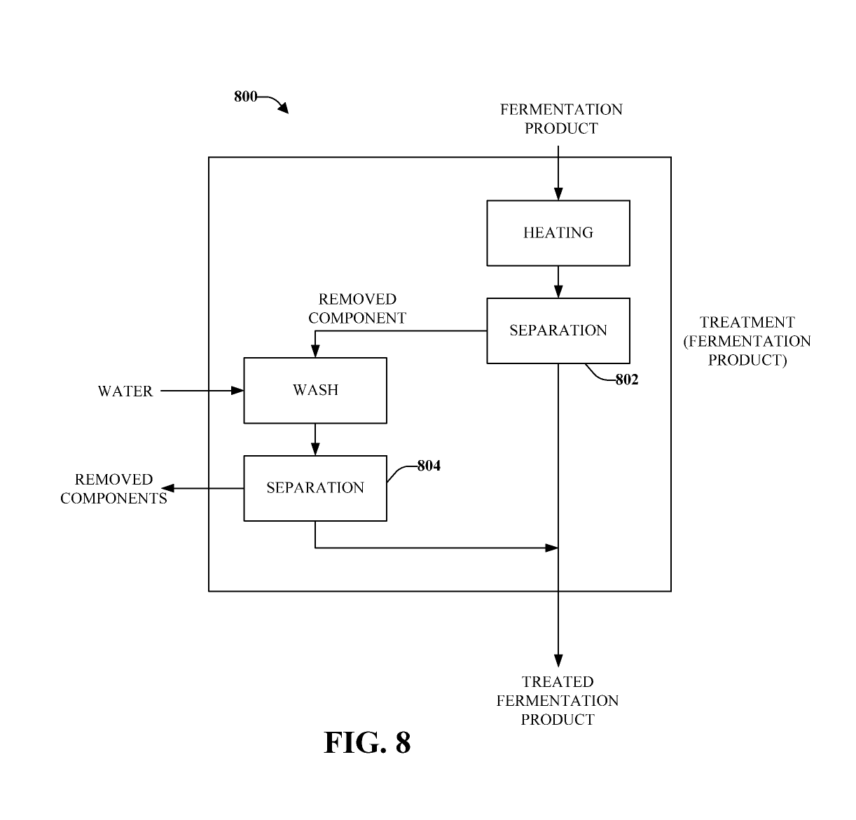

Referring to FIGS. 7A, 7B, and 8, a treatment system 700, 702, 800 for the fermentation product is shown according to exemplary embodiments. The fermentation product is produced in the fermentation system by use of the ethanologen (e.g. yeast cells) to convert by fermentation into ethanol the sugars (e.g. C5 sugars and/or C6 sugars) made available from the biomass (e.g. from treated biomass). The fermentation system may operate in a batch (e.g. fed batch), continuous flow, or other arrangement. The fermentation product (which may also be referred to as or as comprising beer or fermentation broth) will comprise ethanol and water, as well as unfermented matter (e.g. any unfermented sugars) and non-fermentable matter (e.g. residual lignin and other solids), depending upon the composition of the treated biomass supplied to the fermentation system and the treatment applied to the liquid component and the solids component before fermentation; the fermentation product will also comprise in the form of particulate matter (e.g. contained solids) the ethanologen (e.g. yeast cells) that was used to produce ethanol; the fermentation product may also comprise other components produced by the fermentation system, for example, such as glycerol (a product of fermentation).

Enzyme hydrolysis using a cellulase enzyme formulation will generally not break down the lignin in the solids component. Lignin will comprise a substantial constituent of the residual solids in the treated solids component (e.g. hydrolysate) after enzyme hydrolysis of the cellulose. Lignin is unfermentable with the conventional ethanologen formulation used to ferment glucose into ethanol. When the fermentation product, which comprises lignin and other residual solids, is distilled in a distillation column, the residual solids may adhere to surfaces of the distillation column and other equipment, causing interruptions in the operation of the system by fouling and plugging of equipment. When fermentation product is produced without treatment or removal of lignin and other residual solids, the distillation system may only be able to operate for up to five hours without interruption caused by fouling.

According to an exemplary embodiment, the fermentation product may be treated by heating 704 or cooking prior to distillation, as shown in FIGS. 7A and 7B. Heating the fermentation product may cause agglomeration of the particulate matter that comprises the residual solids (including lignin). The treated residual solids have a lowered tendency to adhere to surfaces, allowing the distillation system to operate continuously without interruption for more than ten days. According to an exemplary embodiment, the fermentation product may be heated to a range of 80 to 94 degrees Celsius. According to a preferred embodiment, the fermentation product may be heated to a range of 82 to 90 degrees Celsius. According to a particularly preferred embodiment, the fermentation product may be heated to a range of 84 to 87 degrees Celsius. The treatment system may comprise a vessel, a heat exchanger or a cook tube or other means for achieving the desired temperature. According to an embodiment, the treated fermentation product is distilled to recover the ethanol.

As shown in FIG. 7A, the treatment system can be used with a process where the C5 stream and the C6 stream are co-fermented 706 (see also FIG. 6A). As shown in FIG. 7B, the treatment system can be used with a process where the C5 stream and the C6 stream are separately fermented 708, 710 (see also FIG. 6B).

Referring to FIG. 8, according to an exemplary embodiment, the heated fermentation product may also be separated in a first separation 802 using a centrifuge to remove at least a part of the treated residual solids. The residual solids that have been removed by centrifugation may be washed with water and further separated in a second separation 804 into a solids component, which will comprise substantially solids matter such as lignin and the ethanologen/yeast cells, and a liquid component, which will comprise substantially ethanol and water. The liquid component from the second separation 804 may be combined with the liquid component from the first separation and be supplied to a distillation system to recover ethanol by distillation and dehydration. The removed residual solids (e.g. removed components) may be used or further treated to be used as fuel (e.g. dried into a solid fuel or processed into methane through anaerobic digestion) or as other bioproducts.

The treated fermentation product is distilled to recover the ethanol. According to an embodiment, the distillation can be continuously operated for at least 24 hours. According to a preferred embodiment, the distillation can be continuously operated for at least 48 hours.

According to an embodiment, the fermentation product comprises a solids component that comprises at least 40 percent lignin. The average particle size of the solids component of the fermentation product is typically about 8 to 15 micrometers. According to an exemplary embodiment, fermentation treatment may increase the average particle size of the solids component by at least 50 percent. According to a preferred embodiment, the treatment may increase the average particle size of the solids component by up to 100 percent.

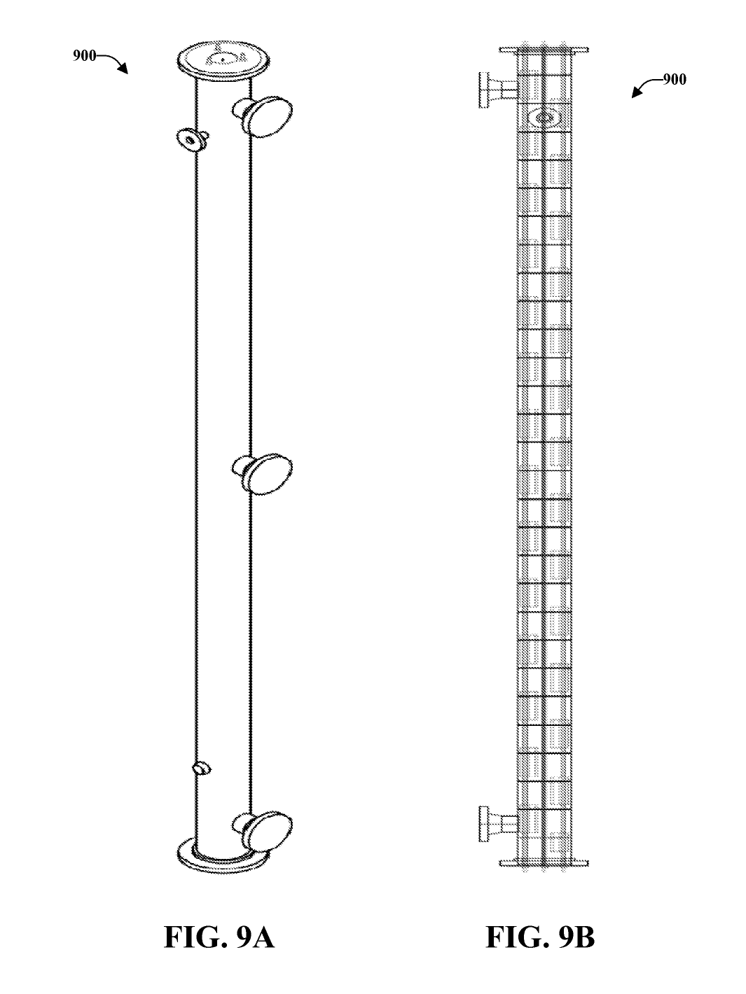

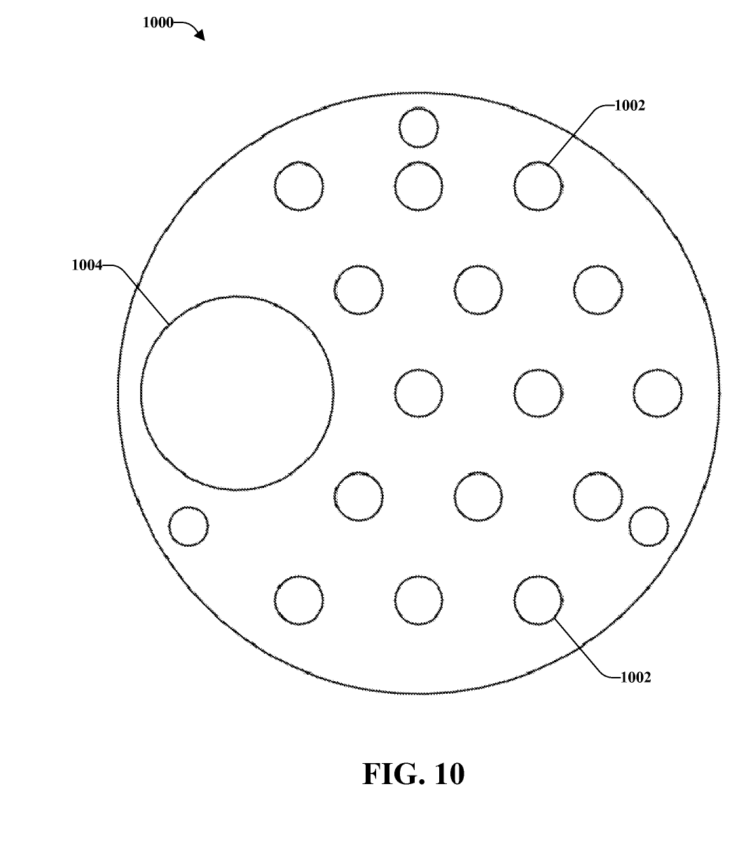

Referring to FIGS. 9A and 9B, a typical distillation column 900 is shown. Any one of various distillation column configurations known in the art could be used. In an exemplary embodiment, the dimensions of the distillation column are approximately 6 inches in diameter and approximately 14.5 feet in length. Multiple distillation trays 1000 (or sieve trays), as shown in FIG. 10, are stacked on the inside of the column. A typical sieve tray comprises multiple small holes 1002 (e.g. sieve holes) that allow steam to move upward and few larger holes 1004 (e.g. down comers) that allow the fermentation product to move downward within the distillation column. The larger holes may employ a tube that protrudes about 0.5 to 1 inch above the tray and about 4 to 8 inches below the tray. The fermentation product is supplied to the distillation column through an opening near the top of the column. Pressurized steam is supplied through and opening at the bottom of the column, heating up the fermentation product and causing the ethanol to evaporate. The vaporized ethanol escapes from the distillation column through an opening at the top of the column. The remaining stillage (with substantially all ethanol removed) leaves the column through an opening at the bottom of the column.

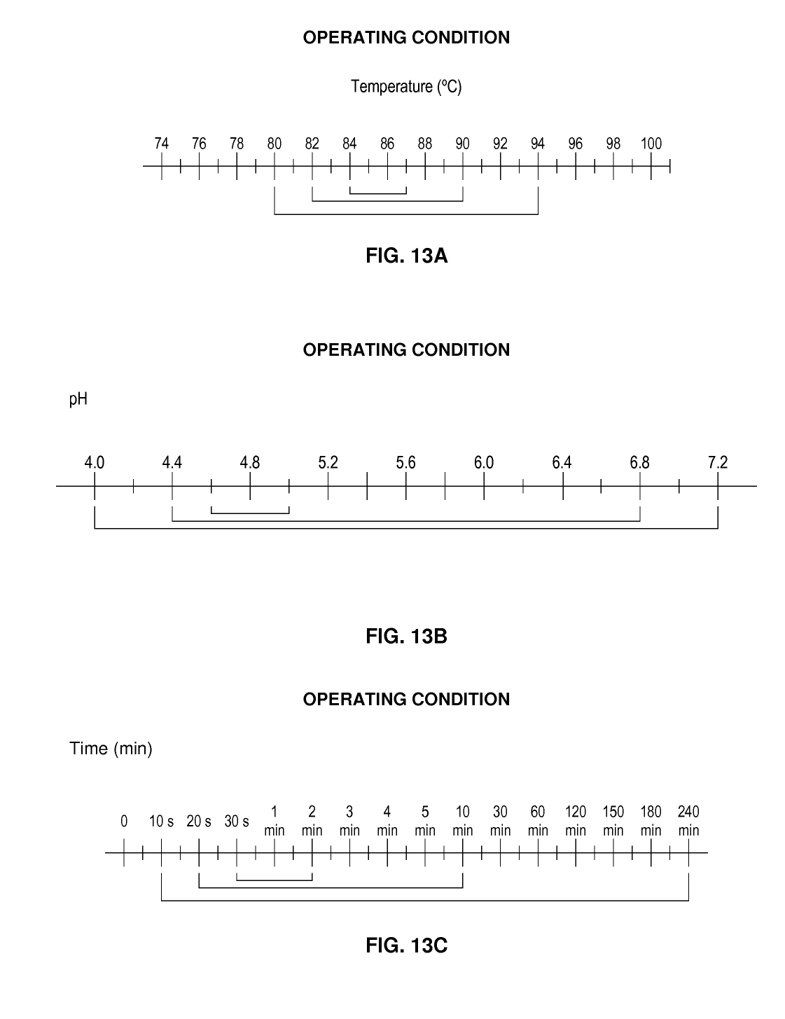

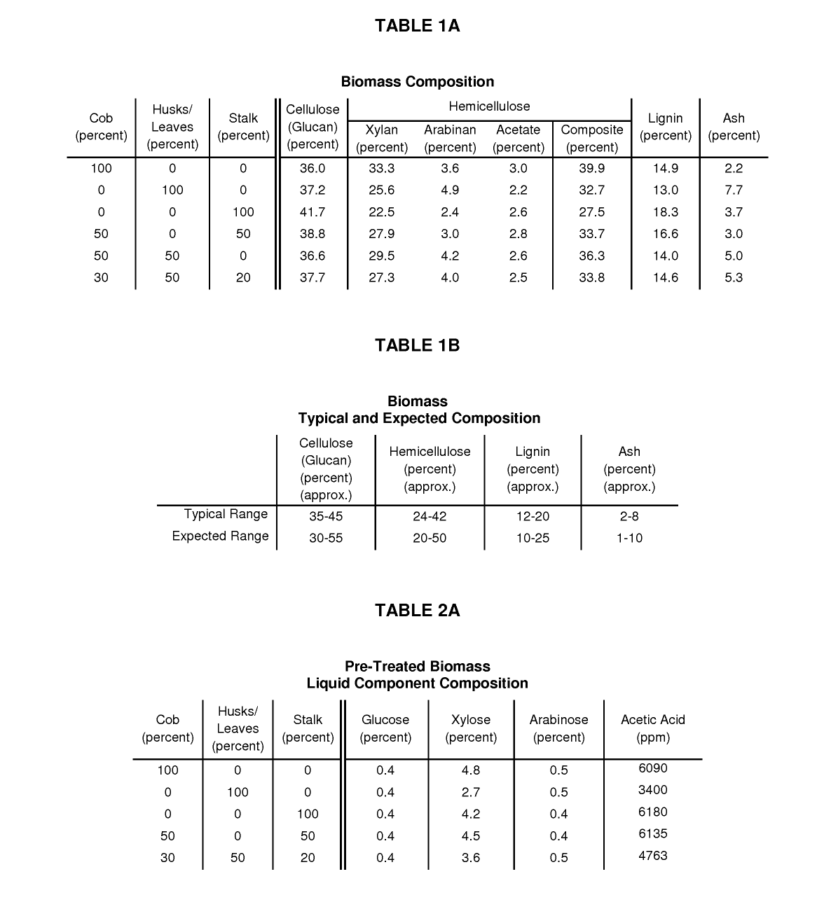

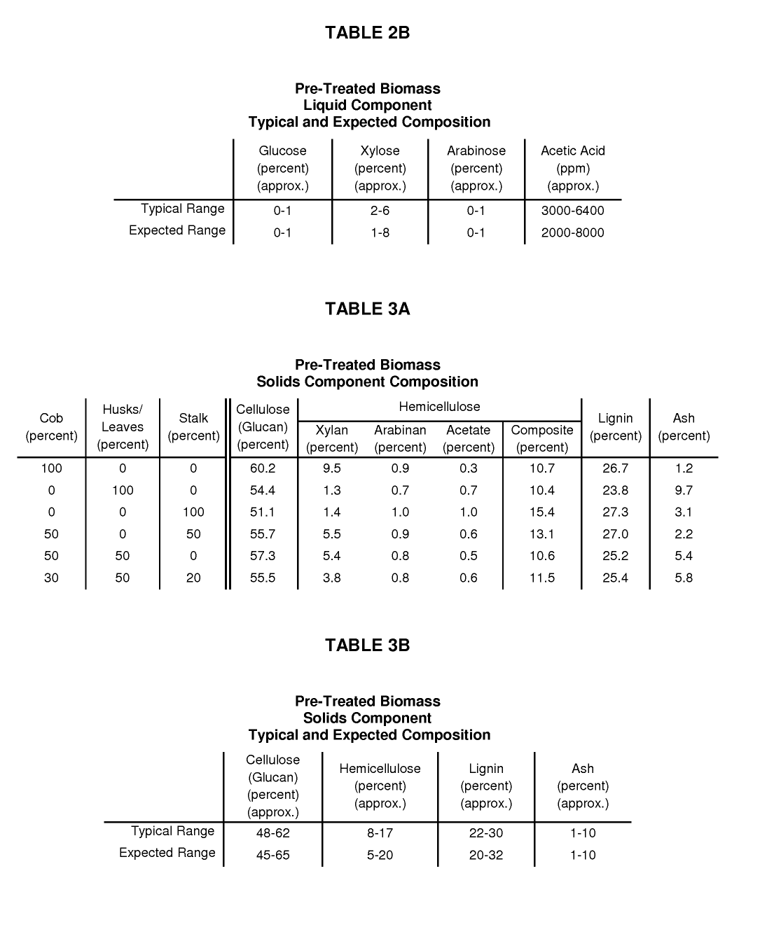

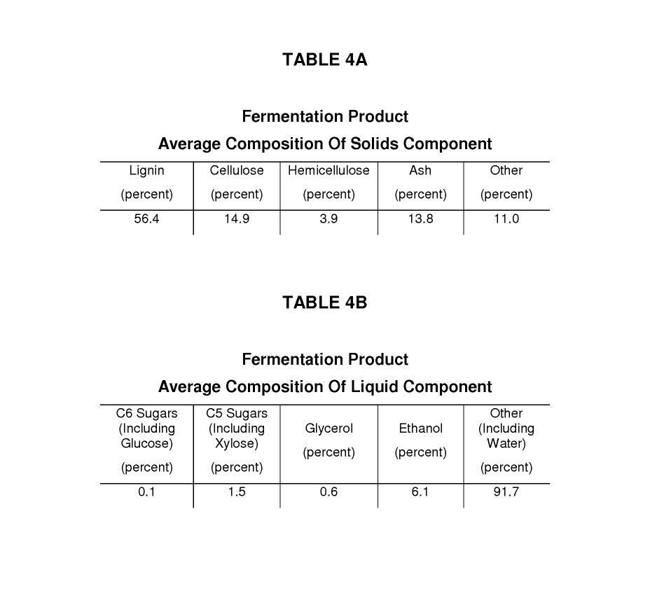

FIGS. 13A, 13B, and 13C show operating conditions for subject parameters for the treatment of the hydrolysate of the solids component of pre-treated biomass to remove lignin according to an exemplary embodiment of the system; operating conditions are shown in the form of nested ranges comprising an acceptable operating range (the outer/wide range shown), a preferred operating range (the middle range shown), and a particularly preferred operating range (the inner/narrow range shown) for each subject condition or parameter.

According to an exemplary embodiment, as shown in FIG. 13A, the temperature during the treatment of fermented biomass may be approximately 80 to 94 degrees Celsius. According to a preferred embodiment, the temperature during the treatment of fermented biomass may be approximately 82 to 90 degrees Celsius, and according to a particularly preferred embodiment, the temperature during the treatment of fermented biomass may be approximately 84 to 87 degrees Celsius.

According to an exemplary embodiment, as shown in FIG. 13B, the pH during the treatment of fermented biomass may be approximately 4.0 to 7.2. According to a preferred embodiment, the pH during the treatment of fermented biomass may be approximately 4.4 to 6.8, and according to a particularly preferred embodiment, the pH during the treatment of fermented biomass may be approximately 4.6 to 5.0

According to an exemplary embodiment, as shown in FIG. 13C, the treatment of fermented biomass may be approximately 10 seconds to 240 minutes. According to a preferred embodiment, the treatment of fermented biomass may be approximately 20 seconds to 10 minutes, and according to a particularly preferred embodiment, the treatment of fermented biomass may be approximately 30 seconds to 2 minutes.

TABLES 4A and 4B show the average composition of the fermentation product. As shown in TABLE 4A, the solids component of the fermentation product may comprise on average about 56 percent lignin (by weight), about 15 percent cellulose, about 4 percent hemicellulose, about 14 percent ash (e.g. inorganic components) and about 11 percent other components. As shown in TABLE 4B the liquid component of the fermentation product comprises on average about 6 percent ethanol, about 0.6 percent glycerol and about 1.6 percent sugars (e.g. C6 and C5 sugars). The solids content of the fermentation product may be approximately 8 to 10 percent by weight, depending on the fermentation conditions. The pH of the fermentation product during treatment and distillation may be in the range of approximately pH 4.6 to 5.0

### EXAMPLE 1

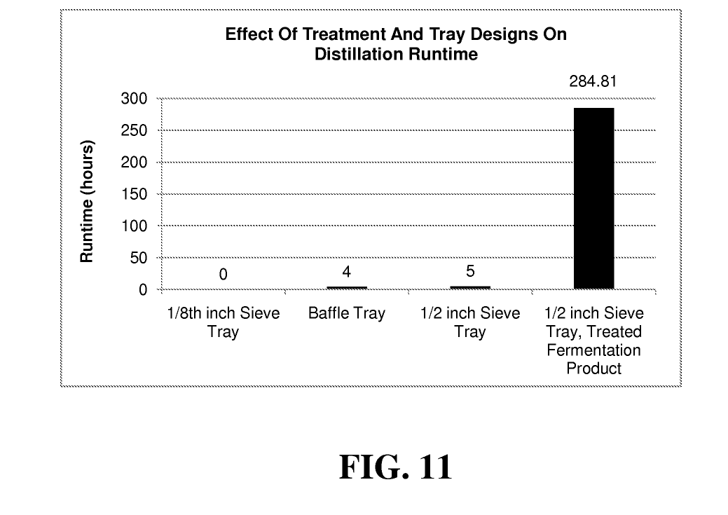

The distillation system as shown in FIGS. 9A and 9B was used in Example 1 to evaluate the effect of different distillation tray designs and fermentation product treatment on distillation runtime. FIG. 11 shows the distillation system run time that was achieved with untreated fermentation product and treated fermentation product. When ⅛ inch hole sieve trays were used in the distillation column, the column could not be operated, and a 0 hour run time was achieved. When baffle trays were used in the distillation column, the column could be operated for about 4 hours. When ½ inch hole sieve trays were used, the column could be operated for about 5 hours. When the fermentation product was treated by heating to 88 degrees Celsius and ½ inch hole sieve trays were used according to a preferred embodiment, improved operability and increased continuous run time of at least 285 hours without fouling and/or plugging of the distillation column were achieved. As shown in FIG. 11, the larger (½ inch) sieve holes improved the operability of the distillation column as compared to the ⅛ inch holes, but were not enough to allow for continuous operation alone.

### EXAMPLE 2

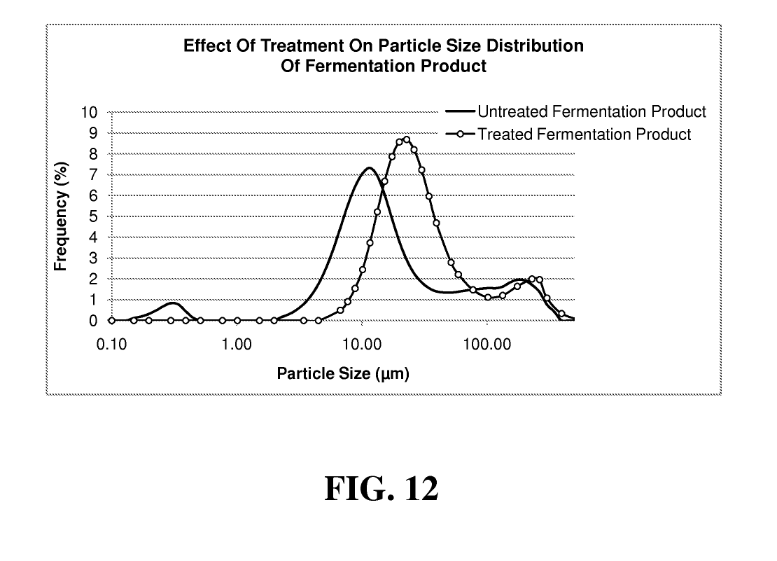

A laboratory experiment was conducted to show the effect of heating on fermentation product as measured by particle size. Fermentation product comprising approximately 9 percent residual solids was prepared by grinding, pre-treating, hydrolysing and fermenting biomass (corn cobs). 200 mL of the fermentation product was heated to 85 degrees Celsius and allowed to cool down to ambient temperature (approximately 21 degrees Celsius). Agglomeration of particles was measured by measuring the particle size before and after treatment using a laser scattering particle size analyzer (available from Horiba, Kyoto, Japan). The particle size distribution of the untreated fermentation product showed peaks at 0.30 micrometers, 11.6 micrometers (the largest peak) and 175 micrometers. The particle size distribution of the treated fermentation product showed no peak around the smaller particle size (less than 1 micrometers), and the other peaks were at 22.8 micrometers (the largest peak) and 229 micrometers. It was observed that the average particle size had increased from approximately 12.8 micrometers before treatment to approximately 24.5 micrometers after treatment. The results are shown in FIG. 12.

The embodiments as disclosed and described in the application (including the FIGURES and Examples) are intended to be illustrative and explanatory of the present inventions. Modifications and variations of the disclosed embodiments, for example, of the apparatus and processes employed (or to be employed) as well as of the compositions and treatments used (or to be used), are possible; all such modifications and variations are intended to be within the scope of the present inventions.

The word “exemplary” is used to mean serving as an example, instance, or illustration. Any embodiment or design described as “exemplary” is not necessarily to be construed as preferred or advantageous over other embodiments or designs, nor is it meant to preclude equivalent exemplary structures and techniques known to those of ordinary skill in the art. Rather, use of the word exemplary is intended to present concepts in a concrete fashion, and the disclosed subject matter is not limited by such examples.

The term “or” is intended to mean an inclusive “or” rather than an exclusive “or.” To the extent that the terms “comprises,” “has,” “contains,” and other similar words are used in either the detailed description or the claims, for the avoidance of doubt, such terms are intended to be inclusive in a manner similar to the term “comprising” as an open transition word without precluding any additional or other elements.

What is claimed is:

1. A method for treating fermented lignocellulosic biomass to be supplied to a distillation system for production of ethanol comprising:
pre-treating lignocellulosic biomass into pre-treated biomass;
separating the pre-treated biomass into a liquid component comprising sugars and a solids component comprising cellulose and lignin;
hydrolysing the solids component of the pre-treated biomass into a hydrolysed biomass comprising sugars and lignin;
fermenting the hydrolysed solids component of the pre-treated biomass into a fermentation product comprising ethanol and lignin;
treating the fermentation product; and
distilling the treated fermentation product to recover the ethanol;
wherein the lignocellulosic biomass comprises cellulose, hemi-cellulose and lignin.

2. The method of claim 1 wherein the treating the fermentation product comprises heating the fermentation product.

3. The method of claim 2 wherein the treating the fermentation product further comprises heating the fermentation product to a temperature of approximately 80 to 94 degrees Celsius.

4. The method of claim 2 wherein the treating the fermentation product further comprises heating the fermentation product to a temperature of approximately 82 to 90 degrees Celsius.

5. The method of claim 2 wherein the treating the fermentation product further comprises heating the fermentation product to a temperature of approximately 84 to 87 degrees Celsius.

6. The method of claim 2 further comprising distilling the treated fermentation product.

7. The method of claim 6 wherein the distilling is continuously operated for at least 24 hours.

8. The method of claim 6 wherein the distilling is continuously operated for at least 48 hours.

9. The method of claim 1 wherein the lignocellulosic biomass comprises at least one of corn cobs, corn plant husks, corn plant leaves and corn plant stalks.

10. The method of claim 1 wherein the lignocellulosic biomass comprises cellulose at about 30 to 55 percent by weight and hemicellulose at about 20 to 50 percent by weight.

11. The method of claim 1 wherein the lignocellulosic biomass (a) comprises corn cobs, corn plant husks, corn plant leaves and corn stalks and (b) comprises cellulose at about 35 to 45 percent by weight and hemicellulose at about 24 to 42 percent by weight.

12. The method of claim 1 wherein the lignocellulosic biomass consists essentially of corn cobs, corn plant husks, corn plant leaves and corn stalks.

13. The method of claim 1 wherein the lignocellulosic biomass comprises cellulose at about 30 to 55 percent by weight, hemicellulose at about 20 to 50 percent by weight and lignin at about 10 to 25 percent by weight.

14. The method of claim 1 wherein the lignocellulosic biomass comprises corn cobs, corn plant husks, corn plant leaves, corn stalks and corn kernel fiber.

15. The method of claim 1 wherein the fermentation product comprises a solids component and wherein the solids component comprises at least 25 percent lignin.

16. The method of claim 15 wherein the fermentation product comprises a solids component and wherein the treating increases a particle size of the solids component by at least 50 percent.

17. The method of claim 1 wherein the fermentation product comprises a solids component and wherein the solids component comprises at least 40 percent lignin.

18. The method of claim 1 wherein the liquid component is combined with the hydrolysed solids component prior to fermentation into the fermentation product.

19. A system for treating fermented lignocellulosic biomass, comprising:
a pre-treatment system that creates pre-treated biomass from lignocellulosic biomass;
a separation system that separates the pre-treated biomass into a liquid component comprising sugars and a solids component comprising cellulose and lignin;
a first treatment system that hydrolyses the solids component of the pre-treated biomass into a hydrolysed biomass comprising sugars and lignin;
a fermentation system that ferments the hydrolysed solids component of the pre-treated biomass into a fermentation product comprising ethanol and lignin;
a second treatment system that treats the fermentation product; and
a distillation system that distills the treated fermentation product to recover the ethanol;
wherein the lignocellulosic biomass comprises cellulose, hemi-cellulose and lignin.

20. The system of claim 19 wherein the second treatment system heats the fermentation product.

21. The system of claim 20 wherein the second treatment system heats the fermentation product to a temperature of approximately 80 to 94 degrees Celsius.

22. The system of claim 20 wherein the second treatment system heats the fermentation product to a temperature of approximately 82 to 90 degrees Celsius.

23. The system of claim 20 wherein the second treatment system heats the fermentation product to a temperature of approximately 84 to 87 degrees Celsius.

24. The system of claim 19 wherein the distillation system is continuously operated for at least 24 hours.

25. The system of claim 19 wherein the distillation system is continuously operated for at least 48 hours.

26. The system of claim 19 wherein the lignocellulosic biomass comprises at least one of corn cobs, corn plant husks, corn plant leaves and corn plant stalks.

27. The system of claim 19 wherein the lignocellulosic biomass comprises cellulose at about 30 to 55 percent by weight and hemicellulose at about 20 to 50 percent by weight.

28. The system of claim 19 wherein the lignocellulosic biomass (a) comprises corn cobs, corn plant husks, corn plant leaves and corn stalks and (b) comprises cellulose at about 35 to 45 percent by weight and hemicellulose at about 24 to 42 percent by weight.

29. The system of claim 19 wherein the lignocellulosic biomass consists essentially of corn cobs, corn plant husks, corn plant leaves and corn stalks.

30. The system of claim 19 wherein the lignocellulosic biomass comprises cellulose at about 30 to 55 percent by weight, hemicellulose at about 20 to 50 percent by weight and lignin at about 10 to 25 percent by weight.

31. The system of claim 19 wherein the lignocellulosic biomass comprises corn cobs, corn plant husks, corn plant leaves, corn stalks and corn kernel fiber.

32. The system of claim 19 wherein the fermentation product comprises a solids component and wherein the solids component comprises at least 25 percent lignin.

33. The system of claim 19 wherein the fermentation product comprises a solids component and wherein the second treatment system increases a particle size of the solids component by at least 50 percent.

34. The system of claim 19 wherein the fermentation product comprises a solids component and wherein the solids component comprises at least 40 percent lignin.

35. The system of claim 19 wherein the fermentation system ferments the liquid component combined with the hydrolysed solids component.
